SEQUENCE LISTING (15 sequences)

SEQ ID NO: 1 - DNA, 24 bp, synthetic construct
  source 1..24: mol_type = other DNA
gagatatacatatggccgaggtgc

SEQ ID NO: 2 - DNA, 20 bp, synthetic construct
  source 1..20: mol_type = other DNA
gcctcttgtctggagtctgg

SEQ ID NO: 3 - DNA, 20 bp, synthetic construct
  source 1..20: mol_type = other DNA
ggcatggacgagctgtacaa

SEQ ID NO: 4 - DNA, 20 bp, synthetic construct
  source 1..20: mol_type = other DNA
cccctcaagacccgtttaga

SEQ ID NO: 5 - DNA, 18 bp, synthetic construct
  source 1..18: mol_type = other DNA
catcaccaccatcaccaa

SEQ ID NO: 6 - DNA, 18 bp, synthetic construct
  source 1..18: mol_type = other DNA
cgttaggaccgtagcgta

SEQ ID NO: 7 - DNA, 52 bp, synthetic construct
  source 1..52: mol_type = other DNA
ttttgtttaactttaagaaggagatatacaatgaaaccagcctttggcacaa

SEQ ID NO: 8 - DNA, 78 bp, synthetic construct
  source 1..78: mol_type = other DNA
tcagtggtggtggtggtggtggccgccctgaaaatacaggttttcgctgccgccttctcttaagagggccaagaattt

SEQ ID NO: 9 - DNA, 35 bp, synthetic construct
  source 1..35: mol_type = other DNA
tatctccaccatcaccaagcttagtacccggaata

SEQ ID NO: 10 - DNA, 30 bp, synthetic construct
  source 1..30: mol_type = other DNA
taggcgactcgacggtcttacgcgttacgt

SEQ ID NO: 11 - DNA, 30 bp, synthetic construct; identical to SEQ ID NO: 10
  source 1..30: mol_type = other DNA

SEQ ID NO: 12 - RNA, 18 nt, synthetic construct
  source 1..18: mol_type = other RNA
ctccttgatgatggccat

SEQ ID NO: 13 - protein, 265 aa, synthetic construct
MKPAFGTNQEDYASYIMNGIIKWGDPVTRVLDDGELLVQQTKNSDRTPLVSVLLEGPPHSGKTALAAKIAEESNFPFIKICSPDKMIGFSETAKCQAMKKIFDDAYKSQLSCVVVDDIERLLDYVPIGPRFSNLVLQALLVLLKKAPPQGRKLLIIGTTSRKDVLQEMEMLNAFSTTIHVPNIATGEQLLEALELLGNFKDKERTTIAQQVKGKKVWIGIKKLLMLIEMSLQMDPEYRVRKFLALLREGGSENLYFQGGHHHHHH

SEQ ID NO: 14 - protein, 180 aa, Campylobacter jejuni
MKEYTLDKAHTDVGFKIKHLQISNVKGNFKDYSAVIDFDPASAEFKKLDVTIKIASVNTENQTRDNHLQQDDFFKAKKYPDMTFTMKKYEKIDNEKGKMTGTLTIAGVSKDIVLDAEIGGVAKGKDGKEKIGFSLNGKIKRSDFKFATSTSTITLSDDINLNIEVKANEKEGGSHHHHHH

SEQ ID NO: 15 - protein, 180 aa, synthetic construct
MKEYTLDKAHTDVGFKIKHLQISNVKGNFKDYSIVIDFDPASAEFKKFDITIKIASVNTENQTRDNHLQQDDFFKAKKYPDMTFTMKKYEKIDNEKGKMTGTLTIAGVSKDIVLDAEIGGMAKGKDGKEKIGFSLNGKIKRSDFKFATSTSTITLSDDINLCWEIKANEKEGGSHHHHHH

(19) United States
(12) Patent Application Publication
(10) Pub. No.: US 2025/0000983 A1
(43) Pub. Date: Jan. 2, 2025

(54) POROUS PROTEIN MICROCRYSTALS AS A SCAFFOLD FOR NUCLEIC ACIDS AND PROTEINS

(71) Applicants: The United States of America as Represented by the Secretary of Agriculture, Washington, DC (US); Colorado State University System, Denver, CO (US)

(72) Inventors: Katherine Eileen Horak, Fort Collins, CO (US); Christopher Snow, Fort Collins, CO (US); Alec Arthur Jones, Fort Collins, CO (US)

(21) Appl. No.: 18/734,755

(22) Filed: Jun. 5, 2024

Related U.S. Application Data
(60) Provisional application No. 63/471,035, filed on Jun. 5, 2023.

(51) Int. Cl. A61K 47/42; A61K 47/64; C07K 14/205; C07K 14/705

(52) U.S. Cl. CPC A61K 47/42; A61K 47/6455; C07K 14/205; C07K 14/705

(57) ABSTRACT

A porous protein crystal drug delivery system, including a nanoporous protein crystal derived from a polyisoprenoid-binding protein from Campylobacter jejeuni (SEQ ID NO: 14) or a variant of a polyisoprenoid-binding protein from Campylobacter jejeuni (SEQ ID NO: 15); and a drug adsorbed to the nanoporous protein crystal, the drug comprising a guest macromolecule selected from the group consisting of a nucleic acid, a protein, and a combination thereof.

The present application claims priority to U.S. Provisional Patent Application No. 63/471,035, filed on Jun. 5, 2023, the contents of which are hereby incorporated by reference in their entirety.

### SEQUENCE LISTING

The instant application contains a Sequence Listing which has been submitted in XML format via EFS-Web and is hereby incorporated by reference in its entirety. Said XML copy, created on Aug. 20, 2024, is named 39247pfs_sequencelisting_corrected and is 147 bytes in size.

### FIELD

The present subject matter relates generally to protein crystals as a delivery vehicle for nucleic acids and proteins.

### BACKGROUND

Nucleic acids are macromolecules that are found in all living cells where they serve as a repository for information. In nanotechnology, nucleic acids have been useful for manipulating material at the molecular level. Designed DNA structures have been used in various biotechnological, pharmaceutical, and nutritional applications. In 1959, mathematician Albert Hibbs and physicist Richard Feynman proposed the concept of “swallowing the surgeon” where tiny robots enter the interior of the body to identify the problem and repair it. R. Feynman, a professor at California Institute of Technology, delivered a lecture called “There is a lot of room at the bottom” where the possibility of building molecular structures was promoted. Following that lecture, the term “nanotechnology” was not used until 1974, by Norio Tanaguchi at an international conference about manipulating a single nanoscale object. This futuristic vision for the medical field was credited to Hibbs and his concept of “swallowing the surgeon.”

In recent decades, these early ideas have become a reality where biomolecular engineering and nanotechnology come together as “Nanobiotechnology”. Nanotechnology use in medical fields has grown significantly in the past few decades for biomarkers, molecular diagnostics, drug discovery, and drug delivery. In particular, nanomedicine is significantly growing to improve quality of life by treating diseases such as cancer and for faster diagnostic methods.

Many nanotechnologies are used in the medical field where drug delivery is an essential part of modern medicine. Nanosized molecular assemblies are capable of carrying therapeutic drugs, DNA/RNA, or proteins. Delivery to the desired targets through biological barriers is important for optimizing the therapeutic load. Nucleic acid-based therapeutics targeting the genetic bases of various diseases for treatment or cure are getting approved by various international counterparts to the Department of Health and Human Services in the U.S.A.

Nucleic acid (NA) payloads might counteract defective genes to achieve therapeutic effect; however, one of the challenges that comes with the use of nucleic acids for therapy is that they are prone to degradation by nucleases. The ideal delivery platform should be able to provide such protection to nucleic acids from various environmental challenges. Through computational methods and protein engineering, it has become possible to provide drug delivery vehicles which encapsulate therapeutic drugs and deliver them to target locations while protecting the drugs from surrounding harsh environments.

The two most common types of nucleic acids are RNA and DNA, and both are formed from nucleotides that contain a five-carbon sugar backbone, phosphate group, and a nitrogen base. However, where DNA contains thymine RNA has uracil. Additionally, RNA contains sugar ribose while DNA has deoxyribose (deoxyribose sugar lacks an oxygen atom). This chemical difference makes RNA prone to hydrolysis, and therefore less stable than DNA. Protection from hydrolysis is important when delivering RNA for therapeutic purposes. It is well-known that RNA is involved in various medical applications such as diagnosis, therapy, and vaccines, such as the Moderna and Pfizer SARS-COVID-19 vaccines. Vaccine platforms of interest involve viral vector-, protein-, RNA-, and DNA-based vaccines in which the process of developing the vaccine includes developing a delivery method. Lipid-based nanoparticles (NP) are a well-known example.

Significant progress has been made in the past few decades on developing nucleic acid-based therapeutics. For example, small interfering RNA (siRNA) has had a demonstrated use for the treatment of fatal diseases or diseases that have limited treatment options. mRNA has also been emerging as a new class of nucleic acid therapeutics that is used in gene therapy. Nucleic acid therapies to treat various genetic diseases have evolved from tools such as transcriptor activator-like nucleases (TALENS), RNA silencing, and clustered regularly interspersed palindromic repeats (CRISPER). Scientists are using these tools to reveal the genetic basis of various diseases such as cancer and type 2 diabetes.

Arguably one of the most promising and high-profile use cases is DNA/RNA based vaccines. During the SARS-COV-2 pandemic, mRNA vaccines were developed by Moderna and Pfizer to protect against infectious diseases such as COVID-19. These vaccines function by causing our cells to produce the SARS-COV-2 spike protein. Generally, many vaccines were developed using various platforms such as RNA (by Moderna and Pfizer) and DNA (by Vaccine Institute and Symvivo), that would deliver genetic materials encoding antigen candidates into the host cell using lipid nanoparticles as a delivery platform. Because of the instability of the naked mRNA and DNA, mRNA and DNA based vaccines require the use of delivery vehicles especially since the mRNA and DNA would have to pass through harsh environments to reach their target. However, lipid nanoparticles are not stable enough to allow oral delivery.

RNA interference (RNAi) could also neutralize target mRNA to inhibit gene expressions and thereby treat diseases. Specifically, RNAs with sequence complementarity to a gene's coding sequence can lead to mRNA degradation which then prevents the translation process of mRNA to protein. RNAi functions by integrating one of the strands of the duplex small interfering RNAs (siRNAs) and turn it into a mRNA-targeting effector complex, also known as RNA-induced silencing complex (RISC). siRNA is typically used for transient gene silencing. Multiple disease targets have been knocked down in vivo using synthetic (siRNA). The generality of the siRNA approach leads to great potential when fighting diseases such as cancer or viral infections. Where abnormal expression or mutation occurs, siRNA is incredibly attractive because it can function in the cytoplasm and does not require nuclear penetration. siRNA strands typically do not exceed 25 nucleotides in length, which is synthetically accessible, making them easily designed and developed.

Although RNA offers great benefits for the purpose of therapeutics and various other applications, RNA structural and sample integrity are critical challenges to address. Compared to DNA, the presence of the 2′ hydroxyl group renders RNA more prone to hydrolysis. RNA is therefore less stable than DNA at room temperature. For therapeutic uses, the integrity of the RNA must be maintained throughout the entire process.

Sample integrity is also hard to maintain while working with RNA because of RNase contamination. RNase is ubiquitous; it can be found on or in human tears, skin, blood, sweat, and saliva. Without the proper usage of aseptic techniques, it is hard to keep RNA strands intact. The rate at which some RNase enzymes degrade RNA is around 39.2 nmol/min per mg (1). Oxidation could also result in RNA degradation through regular atmospheric pollutants that react significantly with RNA. Pollutants are not required but do accelerate the process.

Another route for degradation to occur is through the activity of metallic complexes catalyzing the hydrolytic cleavage of the phosphodiester bond. RNA is typically held at −80° C. for long term storage and −20° C. for short term storage to reduce degradation. However, it has been shown that some ribonucleases maintain activity at −20° C. Therefore, aliquoting RNA samples for single use to avoid thawing-freezing can help reduce degradation. In addition, RNA is very sensitive to pH. Altogether, working efficiently in a clean RNase-free environment while avoiding unnecessary thawing-freezing steps is vital to ensure RNA integrity.

Thus, new drug delivery systems solving the aforementioned problems are desired.

### SUMMARY

The present subject matter relates to a porous protein crystal, or microcrystal, drug delivery system. The porous crystal drug delivery system can include a nanoporous protein crystal and a drug. The drug can be a guest macromolecule selected from the group consisting of a nucleic acid, a protein, and a combination thereof. The nanoporous protein crystal can be derived from a putative polyisoprenoid-binding protein from Campylobacter jejeuni (CJ) (hereinafter, “CJ protein crystal”).

A process of making the porous protein crystal drug delivery system can include preparing protein crystals, adsorbing a guest macromolecule to the protein crystals, and capping the protein crystals after adsorbing of the guest macromolecule. In an embodiment, the protein crystals can be chemically cross-linked prior to adsorbing the guest macromolecule. The method for preparing protein crystals is an optimized process for producing nano-sized crystals from a polyisoprenoid-binding protein from Campylobacter jejeuni (CJ).

### BRIEF DESCRIPTION OF DRAWINGS

Various embodiments will now be described in detail with reference to the accompanying drawings.

FIG. 1 depicts an image of the CJ pores taken using the AFM.

FIGS. 2A, 2B, 2C, and 2D depict SEM images of nanocrystals.

FIG. 3 depicts the location where DpnI cleaves where methylated sites are its recognition site.

FIG. 4 is a graph depicting exemplary qPCR data with variable number of cycles before strong amplification.

FIGS. 5A and 5B depict (FIG. 5A) PYMOL illustration of hexameric D2 domain from N-ethylmaleimide sensitive factor (NSF) (PDB: 1NSF), relative to the CJ major pore. The diameter of the assembled hexamer is ˜110 Å, while the CJ pore is ˜130 Å; and (FIG. 5B) a profile view of NSF cap within the CJ pore; the assembled cap penetrates the pore to a depth of ˜50 Å, or roughly one CJ unit cell.

FIGS. 6A and 6B depict the (FIG. 6A) microfluidic device; and (FIG. 6B) paper pump.

FIGS. 7A, 7B, and 7C depict (FIG. 7A) CJ crystal in its growing condition; and (FIG. 7B) crosslinked CJ crystals in water; and (FIG. 7C) not crosslinked crystals in water (Scale bar=100 μm).

FIGS. 8A, 8B, 8C, and 8D depict SEM images of CJ micro- and microcrystals (Scale bar=1 μm); FIG. 8E is a graph showing size data of crystals as measured from SEM images using ImageJ; and FIG. 8F is a graph showing size data of crystals obtained using tunable resistive pulse sensing (TRPS).

FIGS. 9A and 9B depict loading the crystal with FAM-labeled DNA over time; and (FIG. 9 B) release of FAM-labeled DNA over time using 40 mM ATP.

FIGS. 10A, 10B, and 10C depict (FIG. 10A) a graph showing relative luminescence for Nanoluc-loaded CJ microcrystals, stored at pH 4, 5, or 6, compared with unloaded crystals; (FIG. 10B) time course confocal images showing Nanoluciferase activity in a large crystal, over ˜13 minutes, compared with an unloaded control crystal (scale bar=100 μm); and (FIG. 10C) a graph showing supernatant concentrations of FAM-labeled siRNA, after incubating 300 nM siRNA with increasing concentrations of crystals.

FIGS. 11A, 11B, and 11C depict (FIG. 11A) melt curve analysis demonstrating distinct peaks for amplicons within 5′ and 3′ regions of the 1.8 kbp mRNA; (FIG. 11B) the standard curve for 3′ (upper) and 5′ (lower) amplicons; R²=0.998 for both; and (FIG. 11C) method for relative quantification of hydrolysis, by measuring the ratio of 3′ to 5′ amplicons in reverse-transcribed mRNA from treated and untreated crystals.

FIG. 12 depicts a graph showing that guest RNA was significantly degraded following incubation of loaded CJ nanocrystals in 0.1 mg/mL RNase A for 30 minutes (fold change was calculated using the ΔΔCt method).

FIGS. 13A, 13B, and 13C depict (FIG. 13A) diagram depicting illustration of FRET assay principle; in the absence of hydrolyzing conditions, green fluorescence of FAM is quenched by TAMRA fluorophore at the 3′ end of the molecule; (FIG. 13B) time course series demonstrating hydrolysis of guest DNA, following DNase I activation at 20 minutes; and (FIG. 13C) confocal images for loaded or unloaded crystals which were quenched in either 0.1 M hydroxylamine, or blocked with ˜2 mg/mL SpyCatcher-sfGFP fusion protein, following crosslinking. Scale bars=100 μm.

FIG. 14 depicts in situ FRET-based monitoring of hydrolysis of a single-stranded DNA in the presence of DNase I, for a capped (right) and uncapped (left) crystal (acquisition time is indicated beneath each panel, in mm:ss format; scale bar=100 μm (fluorescence (lighter color) indicates guest hydrolysis).

FIG. 15 depicts a graph showing relative fluorescence values over the entire crystal were obtained over the course of loading and ATP-mediated release (intervals are demarcated by alternating white and gray-shaded regions on the graph; after loading, crystal was washed with increasing concentrations of ATP (0.1 mM to 40 mM)).

FIGS. 16A-16B depict (FIG. 16A) depict images of three crystals over four rounds of loading and release; and (FIG. 16B) depicts a graph of quantitative data demonstrating four cycles of loading and release for three crystals.

### DETAILED DESCRIPTION

The following definitions are provided for the purpose of understanding the present subject matter and for construing the appended patent claims.

### Definitions

It should be understood that the drawings described above or below are for illustration purposes only. The drawings are not necessarily to scale, with emphasis generally being placed upon illustrating the principles of the present teachings. The drawings are not intended to limit the scope of the present teachings in any way.

Throughout the application, where compositions are described as having, including, or comprising specific components, or where processes are described as having, including, or comprising specific process steps, it is contemplated that compositions of the present teachings can also consist essentially of, or consist of, the recited components, and that the processes of the present teachings can also consist essentially of, or consist of, the recited process steps. Said differently, in any descriptions throughout the application of various embodiments using “comprising” language, it will be understood by one of skill in the art, that in some specific instances, an embodiment can alternatively be described using the language “consisting essentially of” or “consisting of”.

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition, an apparatus, or a method described herein can be combined in a variety of ways without departing from the spirit and scope of the present teachings, whether explicit or implicit herein

The use of the terms “include,” “includes”, “including,” “have,” “has,” or “having” should be generally understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present teachings remain operable. Moreover, two or more steps or actions may be conducted simultaneously.

Unless defined otherwise, all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

Using protein crystals as a delivery vehicle for nucleic acids could result in a decrease in their rate of nucleic acid hydrolysis. The first protein crystal was reported in 1840, made of hemoglobin. Since then, nano-scale protein crystals have been used in drug delivery. Protein crystal structures that consist of protein molecules, water, and other chemical molecules (small molecules, ions, etc.) are typically fragile due to their weak mechanical properties; they break or degrade quickly if they are not sustained in a stabilizing solution. Protein crystals are held together by relatively weak intermolecular interactions (hydrophobic interactions, H-bonds, etc.). Thus, biomolecular crystals are weaker and easier to break than non-biological crystals.

In addition to RNA, proteins are another potential therapeutic payload. Over 500 biopharmaceuticals have either been approved or are in advanced clinical trials. Protein crystals could again be an advantageous delivery platform if the crystal lattice is sufficiently porous to allow guest protein adsorption. The payload would be resistant to degradation if the host crystal lattice were to act as a shield against proteinases, and other factors that contribute to degradation.

Crystals also provide advantages of better handling and stability. Additionally, the use of crosslinked protein crystals for delivery of vaccines could increase shelf stability, enable extended release, and/or solve problems related to immunogenicity by slowly releasing drugs. The highly repetitive structure of epitopes in protein crystals could provide enhanced immunogenicity per a study performed by N. St. Clair, et al.

Drug development based on the use of either nucleic acids or proteins has the potential to treat and cure patients. Treatment strategies that attempt to permanently alter a patient's DNA, also called gene therapy, have been pursued to treat genetic disorders and combat cancer. There are tremendous benefits conferred by using RNA therapy as compared to gene therapy. First, messenger RNA (mRNA) has no viral promoters or bacterial sequences which could lead to toxicity. Second, mRNA does not integrate into the host genome. Third, mRNA is safer because gene expression through mRNA is transient compared to DNA. Lastly, mRNA need not pass through the nuclear envelope, which increases the chance of transfecting cells successfully.

While most biomolecular crystals can adsorb small molecules, porous crystals can uptake guest macromolecules, which allows various applications involving drug delivery and biosensing. Protein crystals are also an attractive scaffold from a biodegradability and/or biocompatibility standpoint. Protein crystals have been used successfully in pharmaceuticals (drug delivery, vaccinations, biosensing, etc.) for decades due to their favorable handling, stability, and tunable payload release properties. For example, in the 1930s, protein crystals were developed to deliver insulin which kickstarted the development of microcrystalline structures known as NPH insulin. Crystalline insulin was the first therapeutic crystalline protein to gain approval for use. Crystals are also attractive because of the ease of scaling production and dose release control based on size and shape. In addition, use of crystals to deliver small therapeutic agents as a form of treatment provides advantages, including significant chemical degradation reduction in crystal structures unlike many competing carrier biomaterials. Among all drug delivery materials, crystals are unusually scale invariant in the sense that the nanostructure is the same for tiny or large crystals.

### Porous Protein Microcrystal

The present subject matter relates to porous protein crystal, or microcrystal, drug delivery systems. The porous protein crystal can be a microcrystal or a nanocrystal. The porous protein crystal drug delivery system can include a nanoporous protein crystal, and a guest macromolecule selected from the group consisting of a nucleic acid, a protein, and a combination thereof. In some implementations, a protein-NA mixture may be advantageous. The nanoporous protein crystal can be derived from a putative polyisoprenoid-binding protein from Campylobacter jejeuni (SEQ ID NO: 14) (hereinafter, “CJ protein”) or a variant of the CJ protein (SEQ ID NO: 15) (hereinafter, “CJOpt”). The nanoporous protein crystal can adsorb and retain a guest macromolecule, such as RNA, DNA, and protein. In addition, following adsorption of a guest macromolecule, such as a nucleic acid (NA), the host crystal can be stable while being exposed to nucleases. To protect the loaded macromolecule guest under harsh environmental conditions and nucleases, the pores of the crystals can be obstructed. For example, the pores can be blocked with capping or plugging domains made from DNA or other polymers. In an embodiment, the CJ protein crystals have a particle size ranging from about 100 nm to about 1 mm, or 100 nm to 1 mm. In another embodiment, the CJ protein crystals can have a particle size ranging from about 100 nm to about 10 microns, or 100 nm to 10 microns. In another embodiment, the CJ protein crystals can have a particle size ranging from about 100 nm to about 500 nm, or 100 nm to 500 nm. In an embodiment, the CJ protein crystals can have a particle size below 200 nm, below about 200 nm, or of 200 nm. For the avoidance of doubt, the CJ protein crystals can have a particle size range including any two of such stated endpoints.

In certain embodiments, the CJ protein crystals can possess large pores, e.g., pores of about 10 nm to about 15 nm, about 13 nm, or 13 nm in diameter. As such, the CJ protein crystals according to such embodiments can intake a wide range of guest macromolecules such as DNA, RNA, and proteins.

Crystals on the larger end of the spectrum might be useful for extended release of therapeutics. For delivery in the blood, in certain embodiments any particles would need to be less than 10 microns, about 10 microns, or less than about 10 microns. For extended circulation in the blood, in one embodiment, the particles can have a particle size range of about 100 to about 500 nm would be desired. Additionally, cellular uptake via endocytosis seems to demonstrate an upper size limit of about 200 nm, based on the prevailing literature (DOI: 10.1039/D0CS01127D), so, in one embodiment, the target size for RNA delivery can be below 200 nm, about 200 nm, or below about 200 nm. In some implementations, the nanoporous protein crystal may have a particle size ranging from about 100 nm to about 1 mm. These large crystals may be useful in vaccine deliveries. In other implementations, the nanoporous protein crystal may have a particle size ranging from about 100 nm to about 500 nm. In some implementations, the nanoporous protein crystal may have a particle size of about 300 nm.

One goal of hosting RNA within protein crystals is to advance the oral delivery of RNA to mammals. An embodiment of the drug delivery system described herein can capitalize on the extraordinary promise of RNA as a therapeutic agent. As described herein, the host protein crystals can compensate for the intrinsic susceptibility to degradation of the guest RNA and serve as a capable delivery device with the ability to confer stability and protection.

The amount of guest macromolecule adsorbed by the CJ protein crystals can be dependent on the quantity of crystals present, demonstrating a consistent adsorption per unit crystal mass. To reinforce the CJ protein crystals' ability to protect the loaded macromolecule guest under harsh environmental conditions and in the presence of nucleases, the crystals can be additionally optimized to minimize/prevent hydrolysis within the pores. For example, the pores can be capped with one or more well-fitting proteins and, thereby, prevent the entrance of nucleases. This can increase the ability of the crystal to protect the loaded guest within the pores. In an embodiment, the capping protein is the D2 domain from human N-ethylmaleimide sensitive factor (NSF (SEQ ID NO: 13)). In an embodiment, the capping protein can be functionally modified by, e.g., terminal fusion of a targeting domain or peptide.

Certain exemplary sequences of proteins described herein are listed below in Table 1. For SEQ ID NO: 13, the TEV protease site is double-underlined. The polyhistidine tags are underlined with a single line. CJOpt (SEQ ID NO: 15) is a variant of the CJ protein that includes several mutations intended to stabilize the crystal as well as a surface cysteine for site-specific guest conjugation. The amino acid substitutions specific to CJOpt are double underlined in Table 1. It should be understood that the CJopt crystals have no key differences from CJ crystals for the purposes described herein.

TABLE 1
Primary Sequences of Protein

| | |
|---|---|
| NSF | MKPAFGTNQEDYASYIMNGIIKWGDPVTRVLDDGEL |
| Cap | LVQQTKNSDRTPLVSVLLEGPPHSGKTALAAKIAEE |
| | SNFPFIKICSPDKMIGFSETAKCQAMKKIFDDAYKS |
| | QLSCVVVDDIERLLDYVPIGPRFSNLVLQALLVLLK |
| | KAPPQGRKLLIIGTTSRKDVLQEMEMLNAFSTTIHV |
| | PNIATGEQLLEALELLGNFKDKERTTIAQQVKGKKV |
| | WIGIKKLLMLIEMSLQMDPEYRVRKFLALLREGG |
| | SENLYFQGGHHHHHH (SEQ ID NO: 13) |
| CJ | MKEYTLDKAHTDVGFKIKHLQISNVKGNFKDYSAVI |
| (wild- | DFDPASAEFKKLDVTIKIASVNTENQTRDNHLQQDD |
| type) | FFKAKKYPDMTFTMKKYEKIDNEKGKMTGTLTIAGV |
| | SKDIVLDAEIGGVAKGKDGKEKIGFSLNGKIKRSDF |
| | KFATSTSTITLSDDINLNIEVKANEKEGGSHHHHHH |
| | (SEQ ID NO: 14) |
| CJOpt | MKEYTLDKAHTDVGFKIKHLQISNVKGNFKDYSIVI |
| | DFDPASAEFKKFDITIKIASVNTENQTRDNHLQQDD |
| | FFKAKKYPDMTFTMKKYEKIDNEKGKMTGTLTIAGV |
| | SKDIVLDAEIGGMAKGKDGKEKIGFSLNGKIKRSDF |
| | KFATSTSTITLSDDINLCWEIKANEKEGGSHHHHHH |
| | (SEQ ID NO: 15) |

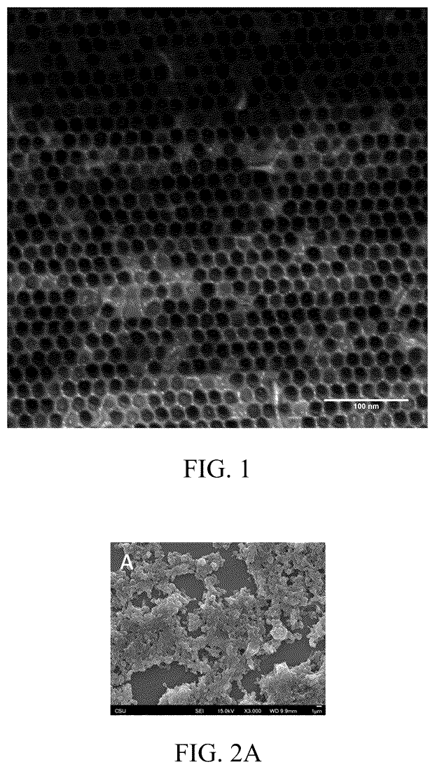

CJ protein crystals can adsorb and retain nucleic acids and enzymes. The unusually large pores can give CJ crystals the capacity to uptake dsDNA. CJ crystals can be highly porous, with nanopores having diameters of about 10 nm to about 15 nm, about 13 nm, or 13 nm. Such nanopores can be arranged hexagonally, resembling the structure of a honeycomb when observed using high resolution atomic force microscopy (AFM) (FIG. 1). Coaxing the protein to crystallize to the right size and crosslinking it to make it stable are not trivial challenges due to the difficulty of precisely controlling nucleation and growth phenomena. Nonetheless, the present compositions and processes successfully achieved a CJ crystal size small enough (about 300 nm, less than 300 nm, or less than about 300 nm) to facilitate delivery in the blood stream of the human body. In addition, crystal stability was dramatically improved with chemical crosslinking, which is important for use in the blood stream. Enzyme adsorption and retention in the pores of CJ protein crystals can provide long term enzyme stability and increased thermal tolerance.

### Process of Making Porous Protein Microcrystal Drug Delivery System

A process of making the porous protein crystal or microcrystal drug delivery system as described herein can include preparing protein crystals, adsorbing a guest macromolecule to the protein crystals, and capping the protein crystals after adsorbing of the guest macromolecule. In an embodiment, the protein crystals can be chemically cross-linked prior to adsorbing the guest macromolecule.

In certain embodiments, the present subject matter relates to a process of preparing the protein crystals as described herein, which can include crystallizing an ultra-pure protein sample using one of three different methods: sitting drop vapor diffusion, hanging drop vapor diffusion, and batch crystallization. In certain embodiments, more than one of said three different methods may be used. Further, the protein sample can include a polyisoprenoid-binding protein from Campylobacter jejeuni (SEQ ID NO: 14) (hereinafter, “CJ protein”) or a variant of the CJ protein (SEQ ID NO: 15) (hereinafter, “CJOpt”).

In an embodiment, crystals having a diameter exceeding 10 microns can be obtained using traditional vapor diffusion methods such as by way of non-limiting example sitting drop vapor diffusion. For example, growth of large CJ crystals has been reported by Kowalski et al. The sitting drop vapor diffusion method involves preparing a reservoir solution including a mixture of varying concentrations of salt, buffer, and precipitant. The precipitant can be a concentrated precipitant. For forming CJ protein crystals, for example, the precipitant can be 3.0 M-3.3 M ammonium sulfate, or about 3.0 M-about 3.3 M ammonium sulfate. The mixture can be pipetted into a middle podium that includes the protein sample. The sitting drop vapor diffusion method can be used to grow large crystals, for example, large crystals that are over 80 μm in diameter.

In an exemplary embodiment, for example for certain biomedical delivery applications, however, it is preferable to grow sub-micron crystals or nanocrystals, such as by way of non-limiting example, crystals having a diameter ranging from about 100 nm to about 1000 nm, or 100 nm to 1000 nm.

In another embodiment, batch crystallization can be used for increased production of smaller crystals, typically microcrystals ranging in size from about 100 nm to about 10,000 nm, or 100 nm to 10,000 nm. Optimized batch crystallization conditions can generate nanocrystals with an average size of about 300 nm, or 300 nm. Batch crystallization can include mixing a larger volume of precipitation buffer with the CJ protein and incubating the mixture for a period of time, such as, by way of non-limiting example, 24 hours, at 4° C. or about 4° C. The precipitation buffer can include, by non-limiting example, 3.3 M ammonium sulfate-3.6 M ammonium sulfate, 3.3 M ammonium sulfate, or 3.6 M ammonium sulfate.

Accordingly, an embodiment of the present teachings is directed to a distinct batch crystallization procedure, in which CJ protein is mixed with a precipitant buffer in 1:5 volumetric ratio, or an about 1:5 volumetric ratio. In an embodiment, the precipitant buffer can include 3.3 M-3.6 M ammonium sulfate, 100 mM Bis-Tris or HEPES, (pH 7.0). After mixing, the resultant crystals can be incubated at a temperature of about 4° C., or 4° C., to slow and stabilize their growth (with constant rotation to prevent localized aggregation or settling), for a period of time ranging from about 18 hours to about 24 hours.

In certain embodiments, the stability of CJ protein crystals outside of their growth conditions/environment can be greatly improved through chemical crosslinking methods. Crystal fragility, for example, can be readily resolved by crosslinking. Therefore, after crystallizing the protein and before loading it with the therapeutic agent, the crystals can be crosslinked in certain embodiments to make them tougher and able to protect guest agents. Such crosslinked protein crystals can withstand solutions with different salt concentrations.

As described herein, crosslinking is the addition of a chemical interaction in the form of strong intramolecular bonds between protein molecules forming a more robust structure. Introducing covalent bonds between adjacent monomers has been shown to potentially greatly improve crystal stability, thus expanding the potential application uses for protein crystals.

In some embodiments, CJ crystals ranging in size from 100 nm to 1000 nm, about 100 nm to about 1000 nm, 1000 nm to 10 μm, and about 1000 nm to about 10 μm can be washed in a high salt buffer to form a mixture prior to crosslinking. Crosslinking can include adding to the mixture a chemical agent such as, by way of non-limiting example, an agent selected from one or more of glyoxal, 1-Ethyl-3-(3-(dimethylamino) propyl)-carbodiimide (EDC), glutaraldehyde (GA), oxaldehyde (OA), and para-Dimethylaminobenzaldehyde (DMAB). The crystals in the mixture can be allowed to crosslink for a period of time ranging from about 1.5 hours to about 2 hours, or 1.5 hours to 2 hours. After crosslinking, the crystals can be looped to a quenching solution for about 1.5 hours to about 2 hours, or 1.5 hours to 2 hours. In certain embodiments, the quenching solution can be selected from hydroxylamine and borohydride. After the quench finishes, the crystals can be washed again, then placed in a storing solution.

As described herein, in one embodiment, CJ protein crystals of about 80 μm size or CJ nanocrystals can be crosslinked with 1% glyoxal, or about 1% glyoxal, followed with 1 M hydroxylamine quench (pH 6 or about 6).

To maximize the production of CJ nanocrystals and to maximize repeatable growth of crystals with effective size control, it may be desired to use a continuous flow crystallization process. For example, a Y-shaped serpentine microfluidic device may be provided to allow rapid consistent solution mixing. The process of growing protein nanocrystals in microfluidic devices could be further automated using a syringe pump and integration of the crosslink and quenching processes. This approach can optimize micro/nanocrystal growth compared to batch crystallization, minimizing aggregation and crystal accumulation which would facilitate downstream biomedical use (e.g., transport in the blood). Preliminary devices made by stacking transparency layers with double-sided adhesive layers that delimit flow channels demonstrated rapid mixing. In certain embodiments, continuous flow of both precipitant and protein solutions should not be limited by crystal size or channel clogging. To this end, modifications can be made to increase channel depth and, thereby, achieve channel dimensions large enough to permit the flow of higher concentrations of micro and nanocrystals.

### Crystal Adsorption, Retention, and Release Capacity

The CJ crystals have the capacity to adsorb, retain, and release guest macromolecules, as described herein. In an embodiment, the loading and release of a guest macromolecule into CJ protein crystals can be facilitated in the presence of metal atoms such as nickel or zinc. Copper, cobalt, or other metals known to bind histidine tags can also work in this role.

For RNA guest molecules, RNA integrity can be first assessed. Assessing RNA integrity is a crucial aspect in research and manufacturing of RNA-based pharmaceuticals and biotechnology. Since RNA is sensitive and prone to degradation, various tests must be performed to ensure non-degraded RNA. One of the most frequent methods used to determine nucleic acid quality is denaturing gel electrophoresis. It is important to be able to determine the precise size of RNA. The benefits of using a classic TAE agarose gel for RNA size determination, by non-limiting example, may include simplicity, speed, and affordability. For proper analysis, RNA samples can be prepared with hot formamide which disrupts RNA secondary structure. By denaturing the RNA, RNA secondary structure can be suppressed, ensuring that the final position in the gel is correlated with RNA length.

Another popular method of quantifying nucleic acids is using quantitative polymerase chain reaction (qPCR). PCR was foundational in molecular biology and is widely used in various applications including mapping genomic sequences. PCR is also the gold standard method to detect bacterial and viral infections such as COVID-19 (by amplifying viral genomic sequences). PCR is a relatively quick process where the results are received within a few hours and is conveniently performed in a laboratory thermocycler. PCR works by first denaturing the DNA at ˜95° C., separating the DNA strands at a slightly lower temperature (e.g., 60° C.) from each other, and producing two single strands instead of one double stranded DNA, where the DNA primers attach to each template DNA. The temperature is then raised (e.g., 72° C.), and the new strand of DNA is made by a polymerase enzyme (e.g., Taq polymerase) generating complementary strands hybridized to each of the original strands. Repetition of the denaturing, annealing, and elongation steps leads to exponential strand duplication. Many repetitions (e.g., 30-40) result in billions of DNA strand copies.

Since the introduction of the PCR protocol, numerous variations have been devised (e.g., RT-PCR, qPCR, and RT-qPCR). Reverse transcriptase PCR (RT-PCR) works primarily with RNA, using reverse transcription to convert RNA to DNA prior to PCR amplification. For the purpose of detection, qPCR, which relies on fluorescence to quantify the growing DNA population, can be used. With RNA being prone to high risk of degradation, RT-qPCR is one optimal, but not the only, procedure used to be able to quantify and detect RNA and be able to assess its integrity.

As described herein, to determine the ability of the CJ crystals to retain and protect RNA from various environmental conditions, many methods can be used. In the non-limiting examples described herein, a TAE agarose denaturing electrophoresis gel was first used to test the size of the RNA after RNase A challenge, with and without adsorption to CJ crystals. Second, a quantitative RT-qPCR assay was developed per a study performed by Padhi et al. to quantify the amount of full-length intact RNA remaining (after elution from host crystals) by comparing two amplicons. Specifically, the relative expression of two amplicons derived from the 3′ and 5′ ends of the RNA strand was measured. This assay relies on the directionality of the reverse transcription reaction, which always proceeds from the 3′ end of the RNA strand to the 5′ end, synthesizing a cDNA strand from the 5′ to the 3′ direction. In theory, if the RNA is intact, the reverse transcription reaction should proceed to completion, generating full length DNA and therefore leading to a ˜1:1 ratio of 3′ and 5′ amplicons. In cases of significant degradation, reverse transcription will yield numerous partial DNA sequences that do not extend to the 5′ amplicon region. In this case, the 5′ amplicon would be lower which would result in a higher 3′: 5′ ratio.

Another alternative quantitative assay was based on Fluorescence Resonance Energy Transfer (FRET) with a 35 mer DNA probe that contained a 5′-terminal FAM (green) and a 3′-terminal TAMRA (red) FRET pair. A description of FRET probe design and application was written by Didenko., V., where a DNA probe of 2 fluorophores within 10 nm of each other can transfer energy at an efficient rate. The idea is that a nucleic acid sequence would contain 2 fluorophores such that one fluorophore donates excitation to an acceptor fluorophore. A dipole-dipole interaction causes the acceptor molecule to excite while the donor is quenched. If the probe oligonucleotide is hydrolyzed, the intensity of the donor molecule would increase over time in exchange for a decrease in the intensity of the acceptor molecule.

Microfluidic devices are an increasingly popular strategy for automating and miniaturizing chemistry and biology experiments. Such devices tend to consist of channels, chips, sensors, and various other features on the μm to mm scale. The devices can provide great benefits of speedy data analysis and significant reduction in reagent volume by integrating multiple components in one device. In addition, the devices can be built for laminar mixing flow. Such devices could be used for detection and diagnosis where they offer the ability to separate molecules and to detect various analytes and antibodies with great sensitivity. Often these devices can be designed and constructed using simple and affordable materials, enabling production of diagnostic devices at low cost. By integrating the components needed for sample processing, fluid handling, signal amplification, and detection in one device without the need for electricity or moving parts, rapid onsite diagnosis could be performed, enabling a higher rate of treatments and health improvements for people in developing countries or natural disaster locations.

As described herein, in one embodiment a device to load and release guest nucleic acid into host protein crystals was developed to measure the release rate of fluorescent labeled nucleic acid by measuring the fluorescence emission intensity of the crystal over time. Microfluidic flow cells were designed and constructed to measure the rate of release of guest NA from CJ crystals. The key design criteria for the device were the ability to maintain a continuous fluid flow and to visualize loading and release in real time.

It was hypothesized that Adenosine triphosphate (ATP) competes with guest DNA for binding sites inside the CJ crystals. To test this hypothesis, precise concentrations of ATP were introduced, release of nucleic acid (NA) was observed in real time, and the rate of release as a function of ATP concentration was measured. ATP is a multifunctional nucleotide which consists of phosphate groups, adenine, and sugar ribose; because of its structure, it provides energy to the cell through breaking the phosphoanhydride bond. ATP also plays an important role in cell functions, membrane transport and degradation of cellular compounds which makes the development of controlled release that is responsive to ATP significant. Also, it was shown in previous studies that ATP can trigger the release of encapsulated cargoes and it is of high interest due to over expressed ATP in certain diseases such as cancer cells and, thus, ATP release of drugs could be of great advantage. By continuously flowing fluorescently labeled NA, the CJ protein crystal adsorbing guest NA in real time was observed.

The present teachings are illustrated by the following examples.

### EXAMPLES

### Example 1

### CJ Protein Expression

For protein expression, Terrific Broth (TB) was prepared by combining 100 mL phosphate buffer salt, 24 g yeast, 4 mL glycerol, and 20 g tryptophan and brought up to 1 L volume using reverse osmosis (RO) water in 2 L flasks. After mixing using shakers at 250 revolutions per minute (RPM) for about 20-30 minutes, the flasks were sterilized in an autoclave (STERIS Amsco lv 250). After sterilization, the media was allowed to sit at room temperature to cool to around 50° C.-55° C. before addition of antibiotics (kanamycin, 50 μg/mL final concentration). A starter culture was prepared by inoculating 10 mL TB media (with kanamycin) from a glycerol stock containing BL21 gold cells previously transformed with the desired plasmid, and incubated at 37° C., 250 RPM overnight. The culture media was transferred to 2 L flasks (10 mL of culture per 1 L of media) and was allowed to incubate at 37° C., 250 RPM. A 1 mL sample was collected every hour to measure optical density (O.D.) using a NanoDrop One Microvolume UV-Vis Spectrophotometer (Thermo Fisher Scientific®) to determine when the liquid culture has reached its optimum exponential phase. After reaching the exponential growth phase protein expression was induced, before hitting the stationary phase.

Specifically, protein expression was induced once the cultures reached an OD between 0.7 and 0.85 using isopropyl β-D-1-thiogalactopyranoside (IPTG), an allolactose mimic, at a concentration of 0.4 mM. This was the induction method selected as all plasmids were designed to include a lac operon controlling expression of the desired protein. Following induction, the cultures were incubated at 25° C., 250 rpm for at least 16 hours. Following protein expression, the cultures were transferred to 1 L cylindrical containers and then centrifuged for 20 minutes at 10,000 relative centrifugal force (RCF) to pellet the cell mass. The pellets were weighed by difference and recorded. Following centrifugation, the cell pellet was resuspended in 80 mL of lysis buffer (50 mM HEPES, 500 mM NaCl, 20 mM imidazole, 10% glycerol, pH 7.4) per liter of culture (˜4 mL/1 g of cell paste). The resuspended pellet was transferred to a glass beaker and was allowed to sit on ice water and cool for about 5-10 minutes. The resuspended cells were sonicated at 50% power for 10 seconds “ON”/30 seconds “OFF” (10-minute process time) while on ice water. After sonication, the lysate was transferred to a 50 mL conical tube and centrifuged at 10,000 g, 4° C., 20 minutes twice or until clarified. The lysate was then run on a Ni-NTA (Nickel-nitrilotriacetic acid) metal affinity column, which is designed to purify His-tagged recombinant proteins. Exemplary poly histidine tags included:

```
Met His His His His His His Xxx Xxx Xxx . . .
Start codon | CAT/CAC | protein specific sequence
                              | Histidine codon |
OR:
Xxx Xxx Xxx . . . His His His His His His
protein specific sequence | CAT/CAC | STOP codon
                                                        | Histidine codon|
```

The nickel ions on the column resin bound to proteins containing poly-histidine tags, which allows the protein to be bound, washed, and later eluted with elution buffer (50 mM HEPES, 500 mM NaCl, 10% glycerol, 500 mM imidazole, pH 7.4) which outcompetes the nickel-histidine interactions, allowing the protein to come off the column.

For the column, the clarified lysate was passed over the resin in a 50 mL glass column and allowed to drip slowly through the resin. Following loading the resin was washed three times with lysis buffer. After washing, elution buffer was applied to the column and collected in three fractions. “Elution #1” included the mixture of elution buffer and lysis buffer while “Elution #3” included whatever was left of protein in the column after the second elution. Synthesis of CJ nanocrystals requires extremely pure protein: therefore, the quantity of the yield was sacrificed for higher purity. Accordingly, the second of the elution fractions was dialyzed overnight into storage buffer (10 mM HEPES, 500 mM (NH₄)₂SO₄, 10% glycerol, pH 7.4) using snakeskin dialysis tubing (10k MWCO). After the dialysis, the dialyzed CJ protein was transferred into a 50 mL conical tube and was purified further via ammonium sulfate precipitation. Briefly, three volumes of 4 M ammonium sulfate were added to the dialyzed protein and incubated with gentle rocking at room temperature for 30 minutes. The sample was then centrifuged at 10,000 g for 15 mins at 25° C., and the clarified sample was filtered through a 0.2 μm cup filter. The protein was then concentrated and desalted with Ultra-15 Amicon® (Merck Millipore #R1JB06172) filters, then diluted with an AS buffer to a final concentration of at least 15 mg/mL. The concentration of the protein was measured via the Coomassie Plus Protein assay (Thermo Scientific #QI221752) using a microplate reader (BioTek Epoch). The protein was subsequently aliquoted and stored at −80° C.

### Example 2

### Protein Crystallization

Crystals were synthesized using both sitting drop vapor diffusion and batch methods. The components required to perform crystallization are precipitant, buffer, and protein. The sitting drop vapor diffusion was performed in 24 well plates and 96 well plates. The mixing process was done using a Gryphon, an automated crystallization robot, to screen conditions for crystal size optimization. The precipitant salt used was ammonium sulfate, with a concentration ranging from 2.5 M to 3.6 M. The buffer was 0.1 M Bis-Tris, with a pH range of 6.5-7.5. Precipitant buffer and WT CJ protein (50 mg/mL) were combined with ratios ranging from 25/75 to 50/50% (protein/buffer) in a total volume of 2 μL and, after sealing the wells, plates were incubated at room temperature for at least 24 hours. Conditions were adjusted to optimize crystal sizes; crystallization of narrower conditions aiming for the desired size was achieved on a 24 well plate with precipitant salt concentration ranging from 3.2 to 3.5 M.

In addition to using the sitting drop vapor diffusion method, batch crystallization was used to produce greater quantities of microcrystals, or nanocrystals as small as (˜300 nm). A precipitation buffer including 3.3 M ammonium sulfate, 100 mM Bis-Tris, pH 6.5, and 2.4% PEG 6000 was prepared, vortexed, and then filter sterilized. Five volumes of precipitation buffer were mixed with 1 volume of concentrated WT CJ protein (30-35 mg/mL), to a final volume of 240 μL. The sample was then incubated on a rocker at 4° C. for 24 hours.

Crystals grown using both methods were chemically crosslinked using glyoxal. Sufficiently large crystals (>30 μm) that grew in the crystal plates were looped and washed 3 times with 4.2 M TMAO (pH 7.5), then transferred to a crosslinking solution containing 875 μL 4.2 M TMAO (pH 7.5), 100 μL 250 mM DMAB, and 25 μL 40% glyoxal for 30 minutes to 2 hours, depending on the size of the crystals. After the crosslink, the crystals were looped to a quenching solution (1 M hydroxylamine, 100 mM DMAB, pH 6) for a similar length of time. After quenching, the crystals were looped and washed 3 more times with water and stored short-term at 4° C.

The microcrystals were pelleted by centrifugation (2,600 g for 3:00 min). After removing the supernatant, the crystal pellets were resuspended in 4.2 M trimethylamine-N-oxide (TMAO), pH 7.5 by light vortexing. Crystals were allowed to soak for 5 minutes, then centrifuged and resuspended in fresh TMAO. These washes were repeated at least two times. After washing, the crystals were resuspended in a crosslinking buffer (4 M TMAO, 25 mM p-dimethylaminobenzaldehyde (DMAB), 1% glyoxal), and incubated on a rotator at room temperature for 1 hour. The crystals were then quenched in 1 M hydroxylamine, pH 5.0, with 100 mM DMAB, for 30 minutes. Following quenching, the crystals were resuspended in a microcrystal storage buffer (50 mM HEPES, 10% glycerol, pH 7.5) and stored at 4° C. long-term.

### Example 3

### Size and Concentration Determination of CJ Microcrystals

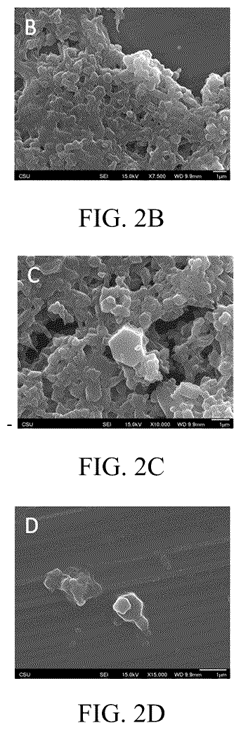

Scanning electron microscopy (SEM) was performed on a JEOL 6500 scanning electron microscope. Crystals were deposited on an aluminum substrate and dried under vacuum, gold sputtered to 10 nm thickness, and imaged with 15 kV beam. Dynamic light scattering (DLS) and zeta potential were measured using a Zetasizer Nano ZS (Malvern Panalytical) (FIGS. 2A-2D).

### Example 4

### Measurement of Guest Loading in CJ Microcrystals

For loading experiments, CJ microcrystal concentrations were determined using tunable resistive pulse sensing (TRPS), and concentrated microcrystals were added to fluorescently labeled DNA, RNA, or plasmid DNA, and incubated for 15 minutes at room temperature. The crystals were then pelleted by centrifugation at room temperature (2,600 g for 3:00 min), and the supernatant was pipetted into a fresh microcentrifuge tube, then spun again at >20,000 g for 5:00 minutes. The concentration of remaining DNA and RNA in the supernatant was then measured on a plate reader and compared to a standard curve. Plasmid DNA was instead quantified by measuring absorbance at 260/280 nm. All measurements were performed in triplicate.

### Example 5

### DNA Synthesis and In Vitro Transcription

To obtain linearized template DNA, plasmid DNA was mini-prepped (OMEGA E.Z.N.A. Plasmid DNA Mini Kit 1 #D6943-02) and the plasmids were concentrated and eluted in nucleus-free water. The DNA concentration was quantified via NanoDrop One (Thermo Fisher Scientific). The plasmids were subsequently stored at −20° C.

Following the miniprep, a pair of designed forward and reverse primers were used to amplify a 2,000 bases long DNA template in a set of 50 μL reactions using a thermocycler MasterCycler Pro made by Eppendorf to perform Polymerase Chain Reaction (PCR). The PCR consisted of 2 steps containing 3 phases each. Prior to the temperature cycling required for annealing and extension, the mixture was heated to 98° C. for 30 seconds to allow for denaturation. Following this initial denaturation, 20 cycles were administered consisting of the following steps: 10 seconds at 98° C., a 20 second 65° C. annealing period—this temperature was decreased by 1° C. for each repetition—and a 2-minute extension at 72° C. Following the final extension, a second round of temperature cycling was performed with a 10 second denaturation at 98° C., followed by 45° C. for 20 seconds, then 72° C. for 2 minutes. This was also cycled 20 times. A crude estimate for the primers' melting temperature can be calculated using the following formula (ThermoFisher: PCR cycling parameters-six key considerations for success):

$${T}_{m} = 4 ( G + C ) + 2 ( A + T )$$

As an alternative method of calculating melting temperature, since the sodium concentration could affect the primer annealing, the following formula provides a more sophisticated approximation (ThermoFisher: PCR cycling parameters-six key considerations for success):

$${T}_{m} = 81.5 + 16.6 ( log log [ {Na}^{+} ] ) + 0.41 ( \% GC ) - \frac{675}{primer} length$$

However, a better melting temperature estimate can be computed using the Santa Lucia nearest neighbor model as made available on the (Tm Calculator|Thermo Fisher Scientific-US) website.

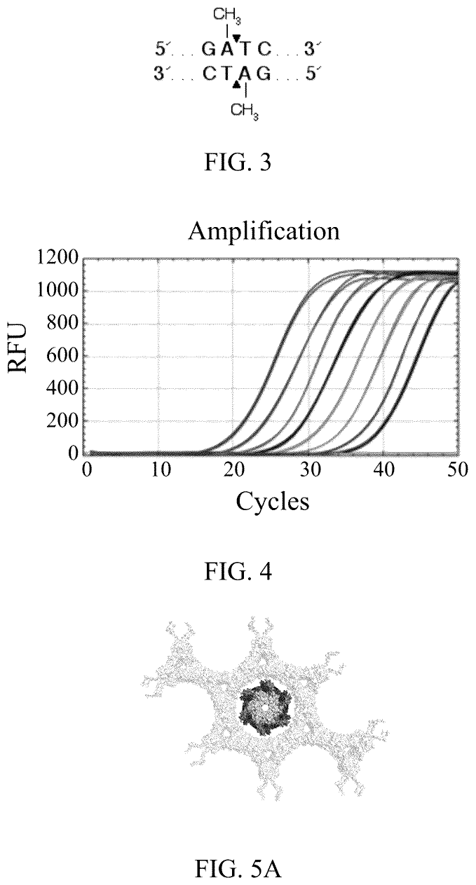

The produced DNA template was then digested using DPN I enzyme from NEB and incubated at 25° C. for at least an hour (FIG. 3). This restriction enzyme cleaves regions of methylated nucleic acid. (New England Biolabs, #R0176L1).

After the digest was done, the DNA was concentrated and washed using washing buffers (PB and PE) from ZYMO Research DNA clean and concentrator kit, then quantified on the NanoDrop One. The length of the sequence was verified using a denaturing 1% TAE electrophoresis gel.

### Example 6

### RT-qPCR Hydrolysis Quantification

A quantitative RT-qPCR assay was developed per a study performed by Padhi et al. to quantify the amount of full-length intact RNA present (after elution from host crystals) using two pairs of primers that synthesize amplicons derived from both ends of the RNA sequence (5′ and 3′). First, the RNA was converted to DNA via reverse transcription, which proceeds exclusively from the 3′ end. The 5′ end of the DNA product was read by the following primers: forward primer (5′-GAGATATACATATGGCCGAG GTGC-3′) (SEQ ID NO:1) and reverse primer (5′-GCCTCTTGTCTGGAGTCTGG-3′) (SEQ ID NO: 2). The 3′ end was read by the following primers: forward primer (5′-GGCATGGACGAGCTGTACAA-3) (SEQ ID NO: 3) and reverse primer (5′-CCCCTCAAGACCCGTTTAGA-3′) (SEQ ID NO: 4). Specifically, the relative expression of two amplicons derived from the 3′ and 5′ ends of the RNA strand was measured.

1.8 kbp RNA (˜10⁻³ M) with CJ nanocrystals (˜300 nm) were incubated overnight at 4° C. for the RNA to load. The loaded nanocrystals were then transferred to two centrifugation tubes (100 kDa MWCO) from Pall Corporation. The crystals were washed three times to remove any excess RNA by centrifugation at 20,000 rcf for 1 minute where the flow-through was collected in a 2 mL tube. This flow-through was then discarded. After the washes, 50 μL TE buffer (1 M, pH 6) was added, with one tube containing 0.1 mg/mL RNase A from OMEGA and the other being a control with no RNase A present. Both tubes were then incubated at 37° C. for 15 minutes. The crystals were then washed again three times to get rid of any traces of RNases with NF water and then incubated with 20 mM ATP for roughly 60 minutes to release adsorbed RNA. The tubes were then centrifuged and the flow-through was added to a reverse transcription (RT) reaction using Invitrogen® SuperScript IV kit. To set up the RT reaction, 1 μL 3′ R primer (10 μM) was added to 10 μL eluted RNA and incubated at 65° C. for 5 minutes. During the primer heat deactivation, RT reaction master mix was prepared by mixing 4 μL 5× buffer, 1 μL RT enzyme, 1 μL RNase Inhibitor, 2 μL 10 mM dNTP, and 1 μL NF per 20 μL reaction where the rest of the 20 μL reaction was RNA template (11 μL). The reaction was incubated at 42° C. for 90 minutes, then heat inactivated for 5 minutes at 70° C.

After the RT reaction was done, an RT-qPCR master mix was made using SYBR Green (Agilent, #600886) where 10 μL of SYBR green was added with (200 nM) of either 3′ or 5′ Forward (F) and Reverse (R) primers, template, and the reaction was upped with nuclease free (NF) water to 20 μL volume per reaction. This brought the number of reactions to 10 where RT-qPCR was also performed on solutions that may or may not have been exposed to RNase A along with contamination control. As set forth in Table 2 using SYBR green as a dye is preferable because the dye does not have a probe-specific design and because it requires less assay setup time and cost.

TABLE 2

| ADVANTAGES | DISADVANTAGES |
|---|---|
| Fluorescent intensity | No target specificity of |
| proportional to the | the dye. |
| amount of PCR | Possibility of |
| products produced. | generation of false |
| Possibility of | positive signals. |
| monitoring the | Recognition of products |
| amplification of any | require melting-curve |
| dsDNA. | analysis. |
| No requirement for | |
| specific probe design. | |
| Decreased assay set-up | |
| time and running costs. | |

A qPCR experiment was also done to test the extent of hydrolysis of dsDNA (135 bp) loaded nanocrystals. Similar steps to the RT-qPCR for the RNA were made where 20 μL DNA (˜10⁻³ M) was incubated with nanocrystals overnight in a 4° C. room. The crystals were transferred to filtered centrifugation tubes (100 kDa MWCO). The crystals were washed three times with 1 M Bis-Tris buffer (pH 6). One of the tubes contained 90 units DNase from Qiagen® and was incubated at 37° C. for 20 minutes. The crystals were then washed with NF water three times to remove DNase by centrifugation at max for 1 minute. After the washes, 20 mM ATP was added and allowed to incubate at room temperature. SYBER Green master mix was made with forward primer (5′-CATCACCACCATCACCAA-3′) (SEQ ID NO: 5) and reverse primer (5′-CGTTAG GACCGTAGCGTA-3′) (SEQ ID NO: 6) using the same format as mentioned in the RT-qPCR using a Bio-Rad CFX Opus 96 Real Time PCR.

For data analysis, Agilent Aria 1.71 and Bio-Rad CFX Maestro software was used to monitor the fluorescence signal (AR) over the number of cycles in real time which produces an amplification plot. Using the amplification plot (FIG. 4) the initial copy number based on threshold cycle (Ct) was quantified. The threshold is inversely proportional to the log of the initial copy number, so the higher the concentration of the template used, the earlier an observable cycle. In addition, these instruments provided the quantification cycle (Cq) which is the needed fractional number of cycles for which the fluorescence reaches a quantification threshold. The ΔΔCₜ method is a standard method to determine the relative changes in gene expression. Essentially, one compares the difference in the threshold cycle (ΔCt) between a target amplicon and the difference in the threshold cycle (ΔCt) for a control amplicon. Here, to quantify RNA degradation, the relative population of partial RNA was compared to full-length RNA by taking advantage of the reverse transcription step. Due to the directionality of reverse transcriptase, most cDNAs, even those generated from hydrolyzed RNA, should still contain most of the 3′ regions from the RNA. However, for those RNAs which have been hydrolyzed, the interrupted sequence means that the 5′ region is unlikely to be reverse-transcribed. As a result, by designing primers to independently amplify a small region in the 5′ or 3′ region, it is possible to assess the extent of degradation of the sample—a ratio close to 1 would indicate minimal hydrolysis, while a ratio close to 0 would indicate nearly complete hydrolysis. Therefore, mRNA hydrolysis can be assessed and compared through multiple treatments regardless of the RNA concentration, by normalizing each treatment to the 3′ copy number.

This type of analysis can be performed using cycle thresholds alone, without the need to compare to a standard curve, so long as the reaction efficiency for the 5′ and 3′ primer sets is between 90% and 110%. The delta-delta Ct method, also known as 2^{-ΔΔCt} method, uses the Ct values given by the thermocycler of each replicate and averages them for each sample. Calculate ΔCt for each sample ΔCt=Ct(treated sample)−Ct(control) where the control was not exposed to DNase/RNase while the treated sample was exposed to DNase/RNase. The control was chosen to be the reference sample to calculate ΔΔCt for each sample ΔΔCt=ΔCt(treated sample)−ΔCt(control). Then finally, from the ΔΔCt, the fold change was calculated 2\hat{ }(−ΔΔCt).

A FRET-based assay was designed and implemented to test the extent of hydrolysis in the guest nucleic acid inside of the crystals because of the difficulty of reliably reproducing the RT-qPCR hydrolysis data. A 30-nt ssDNA probe which contains a 5′ FAM fluorophore and a 3′ TAMRA fluorophore was ordered. In the intact probe, proximity of the TAMRA resulted in quenching of FAM emission (˜520 nm) when illuminated with 488 nm light, resulting in low green fluorescence. In contrast, red fluorescence resulting from FRET would be observable, as the maxima emission is around ˜588 nm. When hydrolysis occurs and the two fluorophores separate, a dramatic increase in the green fluorescence when excited with 488 nm light was observed, due to loss of the quenching effect. CJ crystals were incubated with the probe for 18 hours to load into the pores. The crystals were then transferred to a solution containing (0.1 mg/mL) DNase I buffer and imaged via confocal microscopy. The CJ crystals used were crosslinked with glyoxal followed by either 0.1 M hydroxylamine or ˜2 mg/mL SpyCatcher-sfGFP fusion protein to block the pores to minimize or prevent hydrolysis.

To ensure that nucleases would be unable to diffuse into the crystal pores, a design approach was pursued which relies on physically capping the crystal pores following guest loading. Due to the arrangement of histidine tags in a six-fold radially symmetric manner, a protein which formed a stable hexamer, and which could be engineered such that the histidine tags on the assembled hexamer would align with the tags of the CJ crystal was pursued, to allow the cap to be tethered in a manner similar to past guest proteins. Furthermore, the diameter of the assembled hexamer should be ˜110 Å±10 Å, to ensure that the majority of the pore remains occluded, while remaining slightly undersized to allow it to fit (FIGS. 5A-5B). As a final constraint, the protein should be easily expressed in E. coli and purified using metal affinity chromatography.

Based on these constraints, the D2 domain from human N-ethylmaleimide sensitive factor (NSF) as favorable candidate (PDB code 1NSF) was identified (SEQ ID No:13). As shown in FIGS. 5A and 5B, the dimensions of the assembled hexamer are ideal for obstructing the major pore of CJ crystals. Because the histidine tag was initially too short to fully interact with the histidine tag of the pore wall in a hypothetical 3D model (FIGS. 5A-5B), a TEV protease recognition site was introduced between the protein sequence and the C-terminal histidine tag; this would have the added benefit of allowing rapid uncapping of the crystal. The D2 domain of human NSF (SEQ ID NO:13) was amplified from cDNA from human blood, using the following primers: NSF forward primer (5′-TTTTGTTTAACTTTAAGAAGGAGA TATACAATGAAACCAGCCTTTGGCACAA-3′) (SEQ ID NO: 7) and NSF reverse primer (5′-TCAGTGGTGGTGGT GGTGGTG GCCGCCCTGAAAATACAGGTTTT CGCTGCCGCCTTCTCTTAAGAGGGCC AAGAATTT-3′) (SEQ ID NO: 8). The resulting 828 bp amplicon was assembled in the pSB3 backbone via splice-by-overlap extension PCR. The resulting construct was transformed into BL21-DE (3)-Gold cells. The protein was expressed and purified as described previously.

Two large CJ crystals were crosslinked and soaked overnight in 1 μM hydrolysis probe (5′-TATCTCCACCATCACCAAGCTTAGTACCCGGAATA-3′) (SEQ ID NO: 9). After washing and soaking in 10 mM NiSO₄, one crystal was soaked in 0.5 mg/mL purified NSF for >1 hour at room temperature. Both crystals were then washed and transferred to a new solution containing 0.1 mg/mL DNase I, and imaged directly via spinning-disk confocal microscopy. Immediately after the first frames were acquired, DNase I buffer was added to the drop containing both crystals, which were then imaged over roughly one hour.

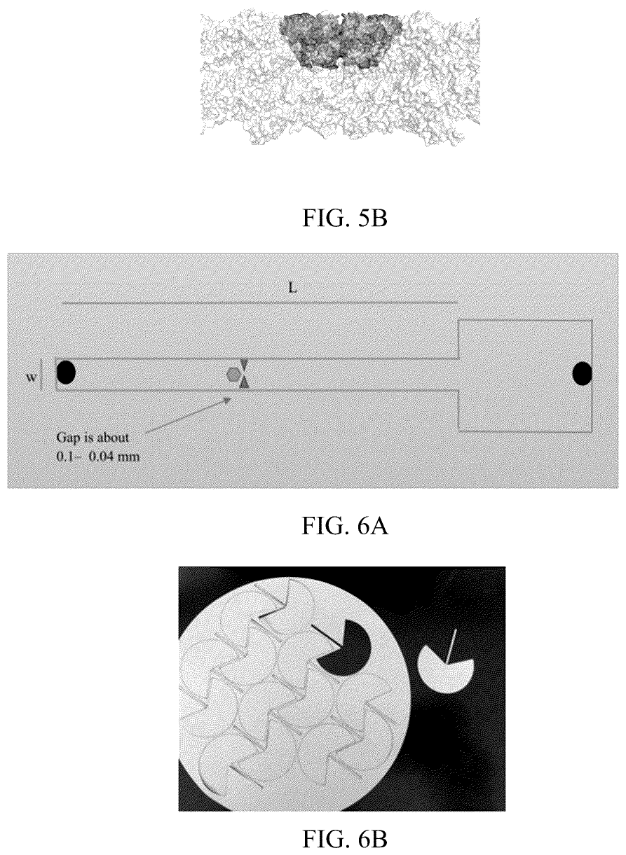

The rate of release of fluorescent labeled nucleic acid from CJ crystals was quantified by measuring the fluorescence emission intensity of the crystal over time by continuously flowing different concentrations of ATP over a period of time using microfluidic devices. The nucleic acid used for this experiment was FAM-labeled ssDNA (5′-TAG GCG ACT CGA CGG TCT TAC GCG TTA CGT-3′) (SEQ ID NO: 10) purchased from Integrated DNA Technologies® (IDT). The microfluidic devices were made of economically affordable material such as transparent polystyrene (3M transparency film for copiers PP2500) and double-sided adhesive (3M 467 MP double sided adhesive). The adhesive and polystyrene were laser cut to obtain a reservoir capacity of 8 μL, and a flow channel height (adhesive thickness) of 50 μm. Width of the flow cell (w) and the diameter of the inlet and outlet ranged between 2 mm-1.4 mm, the dimensions of the reservoir were 12.5×12.5 mm, and the length (L) of the flow cell was 37.5 mm (FIG. 6A). The flow cell contained a blockade with a gap ranging between 0.1 mm-0.04 mm to assist with keeping the crystal stationary. The reservoir has an outlet port where a paper pump (FIG. 6B) was used to sustain fluid flow.

ATP exists naturally in the body. The intracellular concentration of ATP in humans ranges between 1-10 mM. Therefore, the release of the guest nucleic acid from CJ crystals by washing with 4 different concentrations of ATP (100-μM, 2-, 8-, and 40-mM ATP) was tested.

### Example 7

### Growing CJ Protein Microcrystals

CJ protein was concentrated to around 35-50 mg/mL and stored at −20° C. after expression and purification as mentioned previously. Using vapor diffusion sitting drop trials, crystals as large as 400 μm were grown. The Gryphon liquid handling robot was used to screen for the optimum conditions to grow CJ protein crystals to the desired size. It was noticed that crystals grown in the salt concentration range of 3.2-3.6 M provided CJ protein crystals. The closer the concentration to 3.2 M, the larger the crystals.

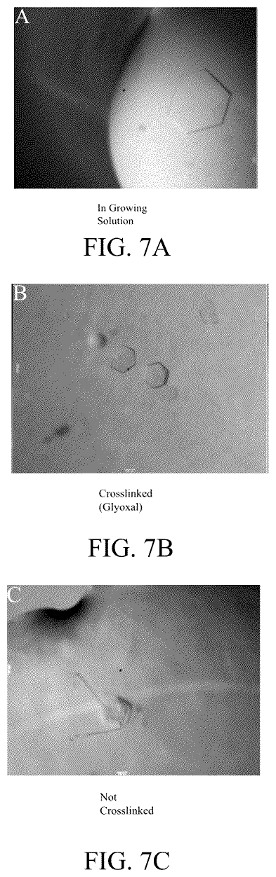

The crystals were generally stable in the environment in which they are grown. As expected, the crystals were quite unstable and would dissolve when moved to a less salty or different pH conditions within seconds (FIGS. 7A-7C).

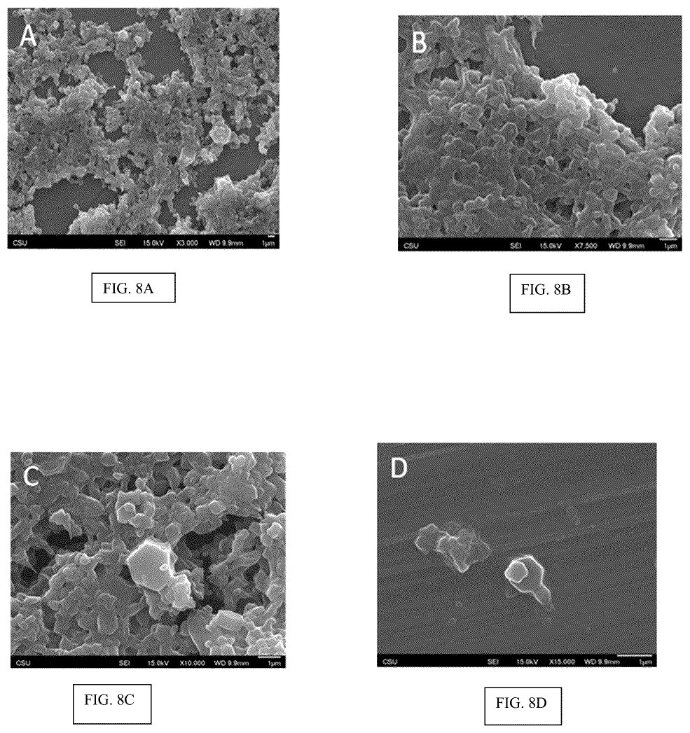

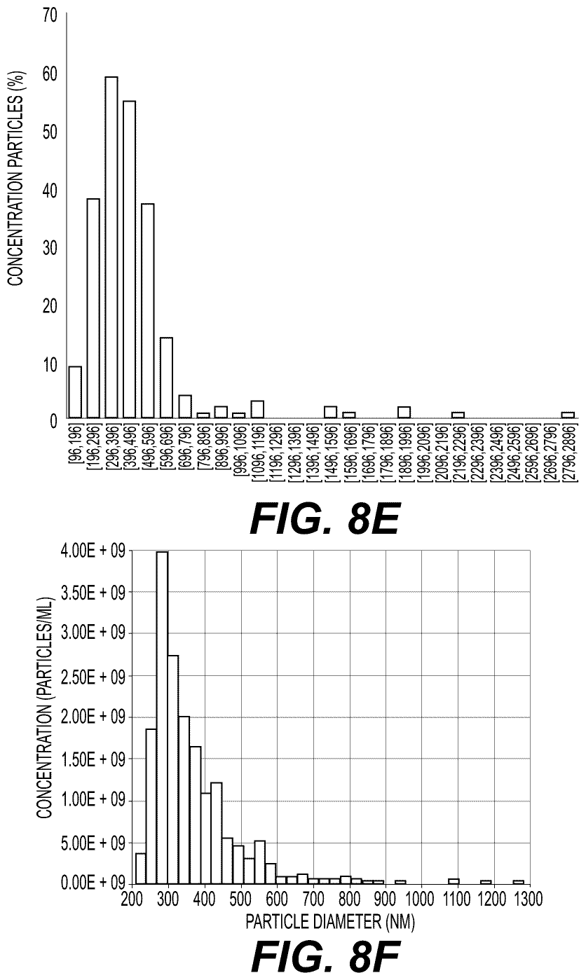

Batch crystallization was performed, and crystal growth conditions were optimized based on pH, precipitant concentration, temperature, PEG, and protein concentration. Of these, protein concentration was found to be a much more significant factor than any of the previous parameters. After characterizing size distribution and concentration of glyoxal-crosslinked microcrystals using tunable resistive pulse sensing, these results were further validated by direct imaging using SEM (FIG. 8A-8D). Crystal sizes obtained from SEM images, as illustrated by the graph in FIG. 15E, were consistent with the TRPS data, as illustrated in FIG. 15F, in terms of the microcrystal size distribution (mean size=367 nm), although the nanocrystal agglomeration was a hindrance towards definitive size characterization. It was difficult to observe the distinctive hexagonal crystal habit until crystal diameters exceeded 500 nm; below this size, CJ nanocrystals appeared to be amorphous.

### Example 8

### Loading and Releasing of Guest Molecules

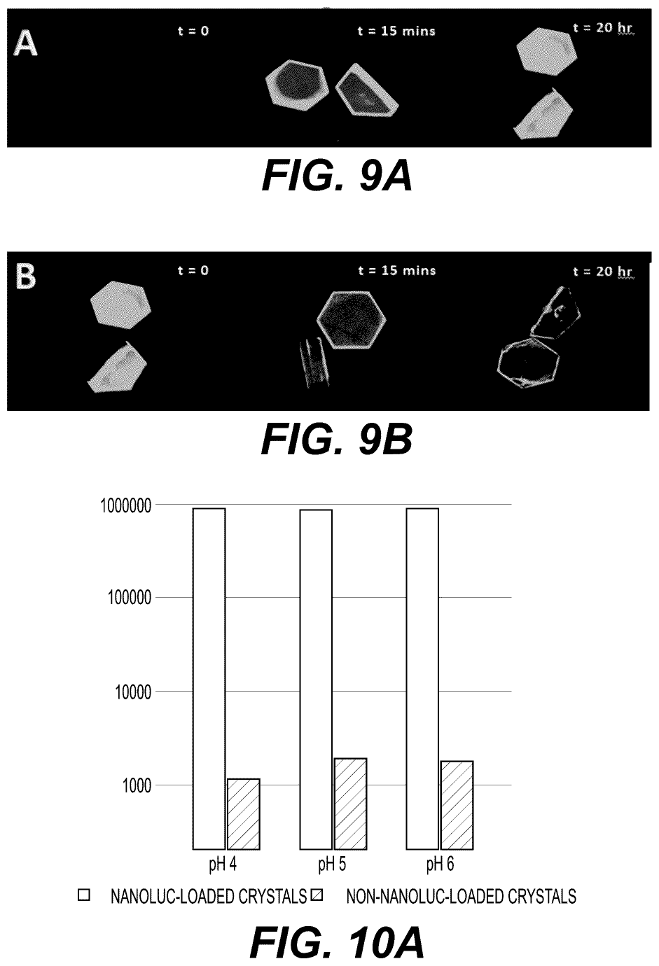

CJ crystals possess large pores (˜13 nm diameter), which allows them to intake a wide range of guest molecules such as DNA, RNA, and proteins. Their ability to load and release guest molecules were first tested. Three CJ crystals were loaded with (1 μM) FAM-labeled ssDNA 30 nt long (5′-TAG GCG ACT CGA CGG TCT TAC GCG TTA CGT-3′) (SEQ ID NO: 11) over 15 minutes and the loading was visualized in real time. The crystals were then left to incubate at about 4° C. overnight to get complete loading (FIG. 9A). The next day, the three CJ crystals were washed with 8 mM ATP where the guest nucleic acid was visualized in a steady release over 15 minutes. The crystals were then left to incubate at about 4° C. overnight in 40 mM ATP to get a complete release (FIG. 9B). However, in a panel at t=20 hours, it could be seen that the borders of the crystal remain fluorescent which could indicate tightly bound FAM-labeled ssDNA remaining inside the crystal or on the crystal surface.

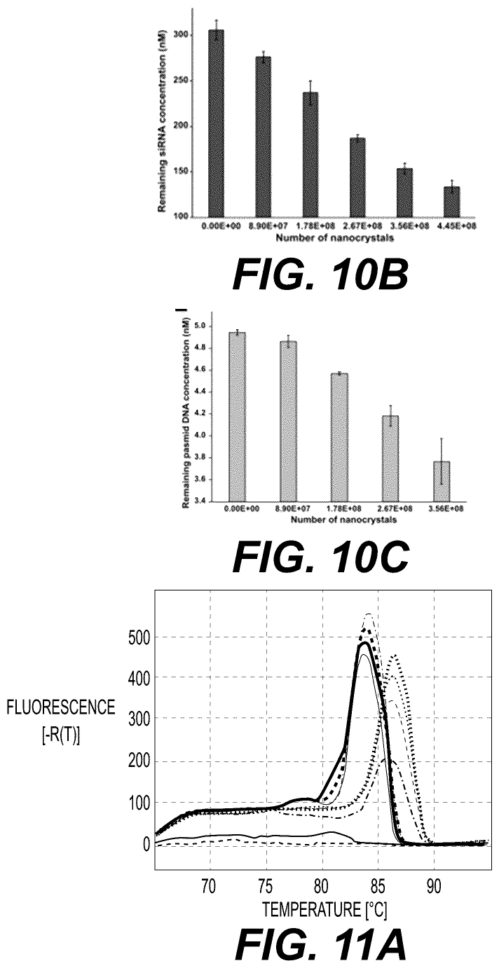

The RNA and DNA adsorptive capacity of CJ microcrystals was tested by incubating increasing concentrations of crystals with fixed concentrations of FAM-labeled, 19-bp anti-mCherry siRNA (300 nM) (5′-CUC CUU GAU GAU GGC CAU G-3′) (SEQ ID NO: 12), or 5.8 kbp plasmid DNA (˜17.9 ng/uL). A linear increase in microcrystal concentration correlated was observed with a linear decrease in supernatant concentration for both species, following a 30-minute incubation at room temperature. From this data, the average number of siRNA or plasmid molecules adsorbed per microcrystal (14,900±720 copies of siRNA, or 353±80 copies of plasmid, per crystal) was quantified. In addition to passive adsorption of nucleic acids, adsorption and functional activity of adsorbed enzyme was demonstrated. Consistent with a loading protocol previously described by Kowalski et al. for polyhistidine-tagged BSA, a large (>100 μm) crosslinked CJ crystal was soaked overnight in a loading buffer (50 mM HEPES, 300 mM NaCl, 10% glycerol, 10 mM nickel sulfate, pH 7.5), then subsequently soaked in 5 mg/mL his-tagged Nanoluciferase. This same loading procedure was performed using CJ microcrystals. By measuring chemiluminescence via plate reader, it was found that the CJ nanocrystals were able to retain guest Nanoluciferase, following multiple washes in acetate or Tris buffers at varying pH values (FIG. 10A). Furthermore, chemluminescence in the large CJ crystal, via confocal imaging, over a ˜12.5 minute window, following addition of 50 μM furimazine, was directly monitored.

For DNA synthesis and in vitro transcription, PNZ108 plasmids were mini-prepped, and a region (2,000 bp) was amplified using PCR. Next, the linear DNA was used as a template to transcribe intact RNA (1,800 bp) and used 1×TAE gel electrophoresis to ensure that the targeted length was obtained. Five PCR reactions were performed to amplify the DNA template of the PNZ108. The five reactions were verified on agarose gel. The samples were then cleaned and concentrated and stored in the −20° C. or 4° C. for short term storage.

With purified 2,000 kb DNA in hand, in vitro transcription was performed to obtain 1.8 kb RNA. The RNA product was verified on a denaturing RNA electrophoresis 1×TAE gel using formaldehyde. Formaldehyde acts as a denaturing agent for RNA during agarose gel electrophoresis, which also helps maintain RNA integrity during gel handling and separation. The rest of the RNA sample was aliquoted into 5 μL aliquots and stored at −80° C.

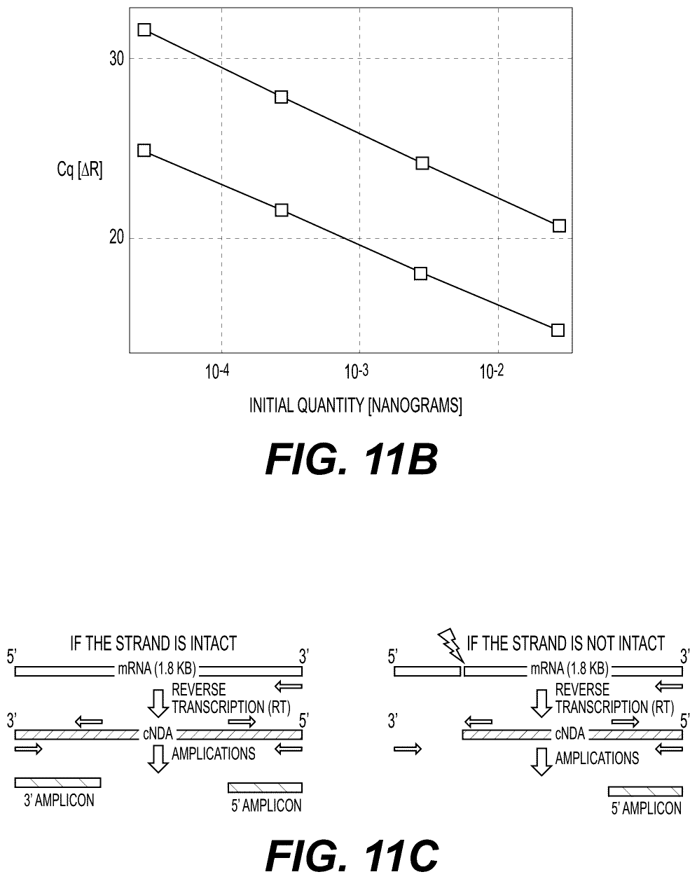

For quantifying RNA hydrolysis in loaded nanocrystals, two pairs of primers were designed for amplifying the 5′ and 3′ regions. A standard curve was performed to test the quality of the primers (FIG. 11A). These primer pairs had close reaction efficiencies (95.4% and 96.0% for 5′ and 3′ amplicons) (FIG. 11B).

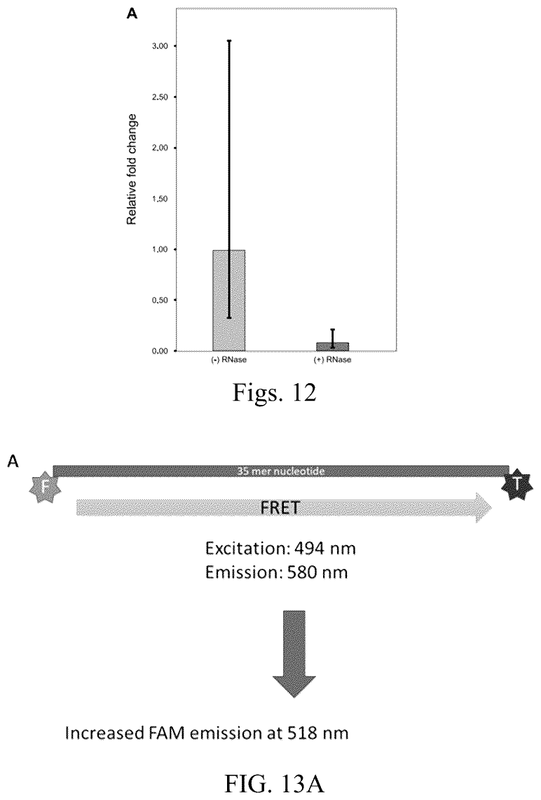

Pairs of primers were designed to amplify small regions (˜150 bp) at opposite ends of the RNA sequence (˜1.8 kbp). Following exposure of nanocrystals loaded with mRNA to nucleases, and RNA release via incubation in 20 mM ATP; the sample RNA was reverse transcribed using the 3′ primer to produce cDNA (of varying size due to the potential hydrolysis). Then, qPCR was used to compare the number of intact 3′ amplicons and the number of intact 5′ amplicons, and to calculate the relative fold change of intact RNA in the sample, using the ΔΔCt method. In other words, using the ΔΔCₜ method, mRNA hydrolysis was measured from the 5′ amplicon quantity relative to the 3′ amplicon quantity (FIG. 11C). After measuring the difference between the 5′ and 3′ cycle thresh holds for each sample (+/−RNase A), the difference in the resulting values was used to determine the fold change by taking the antilog (2^{-ΔΔCt}). A decrease in intact RNA following RNase exposure was observed (FIG. 12), suggesting that the crosslinked nanocrystals were not sufficient to protect guest RNA on their own.

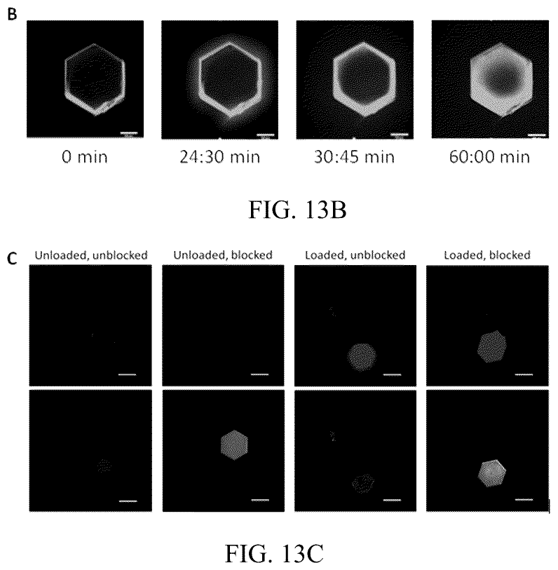

Hydrolysis directly in the crystal was monitored using a FRET-based probe (FIG. 13A) consisting of a 35-nt ssDNA probe that contained 5′ FAM and 3′ TAMRA fluorophores. After loading this probe into a large CJ crystal, the crystal was transferred to a solution containing DNase I buffer and imaged via confocal microscopy. After adding DNase I to a final concentration of 0.1 mg/mL, followed by addition of DNase I buffer after ˜ 20 minutes, a sharp increase was observed in FAM fluorescence within the crystal, indicating guest hydrolysis. Together with the data obtained by RT-qPCR in the RNA-loaded microcrystals, these results indicated that the crystal scaffold alone was insufficient to protect guest DNA or RNA from nucleases.

### Example 9

### Capping Protein

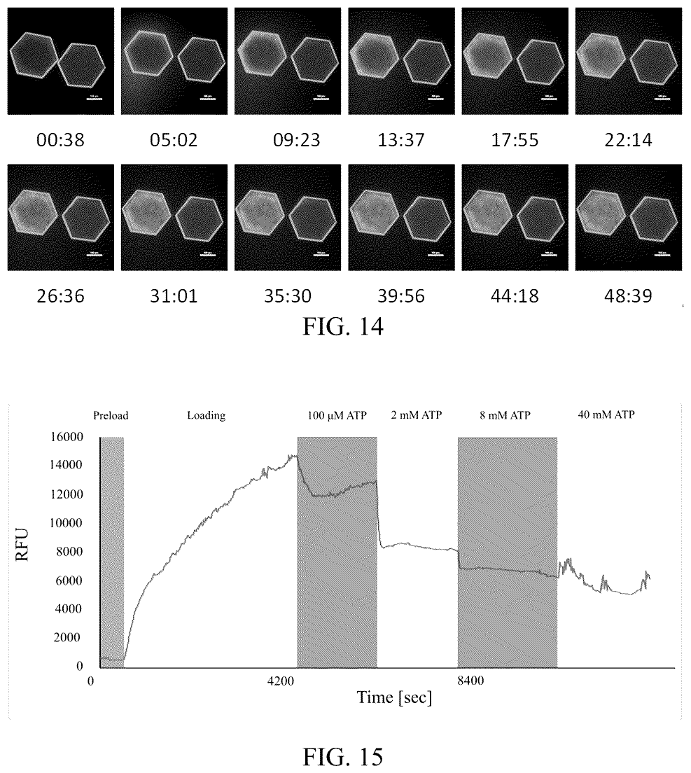

In situ FRET-based monitoring of hydrolysis of a single-stranded DNA in the presence of DNase I demonstrated that CJ pores can be protected by capping proteins. Referring to FIG. 14, fluorescence, indicating guest hydrolysis, was observed in the uncapped crystal (left), but not in the capped crystal (right), indicating that the NSF cap conferred protection against environmental nucleases. These results were replicated in a different pair of CJ crystals.

### Example 10

### Rate of Release

The rate of release of DNA based on ATP concentration from CJ crystals was measured.

A functional cargo molecule is only useful if it can be released. To demonstrate controlled release, an in-house flow cell device was developed to measure intensity over time using fluorescent DNA to show loading and release under the confocal microscope. The flow cell was used to visualize how long it takes to load the CJ protein crystals and release the load using different concentrations of adenosine triphosphate (ATP), below and above the concentration of ATP in the cellular level where it ranges between 1-10 mM (43). Specifically, the CJ protein crystal was washed with a range of different ATP concentrations: 0, 0.1-, 2-, 8-, and 40-mM where 0 mM was Nucleus Free (NF) water (FIG. 15). The crystal was prewashed with 8 mM ATP for about 5-6 minutes then followed by FAM labelled DNA loading for 48 minutes whereupon it was visually completely green, but the intensity was increasing indicating that the loading was still occurring. The crystal was then washed with NF water for about 10 minutes followed by washing with 0.1-, 2-, 8-, and 40-mM ATP for 30 minutes on each concentration. The entire process of releasing the guest DNA was 2 hours long. Surprisingly, the FAM labelled DNA was not completely released even after exposing the crystal to a very high concentration of ATP (40 mM) with a good fraction of the guest DNA remaining in the crystal. In addition, a modest increase in fluorescence following ATP washing was observed. It is believed that, because the crystal was imaged in a single z-plane over time, the initial ATP may have increased the rate of transport of fluorescently bound DNA further into the crystal. Alternatively, the increase in fluorescence may be due to incomplete washing of the crystal following loading. Finally, ATP might authentically increase the affinity of the crystal for the oligonucleotide via an unknown mechanism. In any event, the guest DNA failed to completely release, even after washing with 40 mM ATP for several minutes.

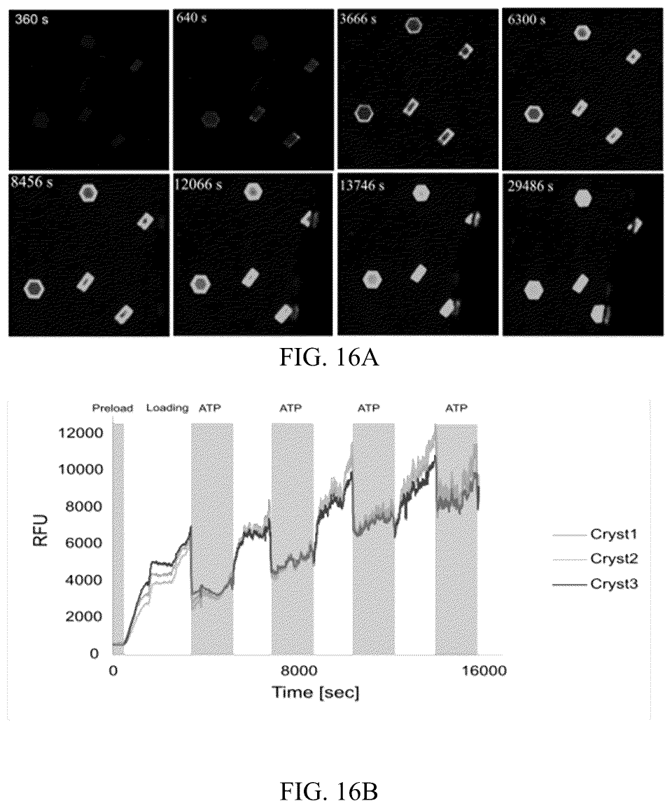

Finally, to assess whether the crystals display hysteresis with respect to guest loading and release, three crystals were monitored over four rounds of loading and release (FIGS. 16A-16 B). It was observed that guest DNA adsorption accumulated over time, owing to incomplete release of guest DNA between each cycle (repeated exposure to 40 mM ATP). It is believed that a fraction of the adsorbed guest is bound much more strongly, while weakly bound DNA was released in the presence of ATP, whereupon it can re-adsorb at a more favorable site following release.

It is to be understood that the porous protein crystal drug delivery system, compositions containing the same, and methods of using and producing the same are not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

We claim:

1. A porous protein crystal drug delivery system, comprising:
a nanoporous protein crystal derived from a polyisoprenoid-binding protein from Campylobacter jejeuni (SEQ ID NO: 14) or a variant of a polyisoprenoid-binding protein from Campylobacter jejeuni (SEQ ID NO: 15); and
a drug adsorbed to the nanoporous protein crystal, the drug comprising a guest macromolecule selected from the group consisting of a nucleic acid, a protein, and a combination thereof.

2. The porous protein crystal drug delivery system according to claim 1, wherein nanopores of the nanoporous protein crystal are obstructed.

3. The porous protein crystal drug delivery system according to claim 2, wherein the nanopores are obstructed with at least one capping protein.

4. The porous protein crystal drug delivery system according to claim 3, wherein the at least one capping protein comprises D2 domain from human N-ethylmaleimide sensitive factor (NSF) (SEQ ID NO: 13).

5. The porous protein crystal drug delivery system according to claim 1, wherein the nanoporous protein crystal has a particle size ranging from about 100 nm to about 1 mm.

6. The porous protein crystal drug delivery system according to claim 5, wherein the nanoporous protein crystal has a particle size ranging from about 100 nm to about 500 nm.

7. The porous protein crystal drug delivery system according to claim 5, wherein the nanoporous protein crystal has a particle size of about 300 nm.

8. A process of making a porous protein crystal drug delivery system, comprising:
preparing nanoporous protein crystals, wherein the process of preparing the nanoporous protein crystals comprises mixing a precipitant with a protein sample;
adsorbing a guest macromolecule to the nanoporous protein crystals, the guest macromolecule being selected from the group consisting of DNA, RNA, and a protein; and
capping the nanoporous protein crystals with at least one capping protein after adsorbing of the guest macromolecule.

9. The process of claim 8, wherein the nanoporous protein crystals are derived from a polyisoprenoid-binding protein from Campylobacter jejeuni (SEQ ID NO: 14) or a variant of a polyisoprenoid-binding protein from Campylobacter jejeuni (SEQ ID NO: 15).

10. The process of claim 8, wherein the nanoporous protein crystals have a particle size ranging from about 100 nm to about 10000 nm.

11. The process of claim 8, wherein the at least one capping protein comprises D2 domain from human N-ethylmaleimide sensitive factor (NSF) (SEQ ID NO: 13).

12. The process of claim 8, wherein the process of preparing the nanoporous protein crystals further comprises crystallizing a protein sample by a method selected from the group consisting of vapor diffusion and batch crystallization.

13. The process of claim 12, wherein the vapor diffusion is selected from sitting drop vapor diffusion and hanging drop vapor diffusion.

14. The process of claim 11, wherein the precipitant is ammonium sulfate.

15. The process of claim 8, wherein the nanoporous protein crystals are crosslinked by a process comprising mixing the nanoporous protein crystals with a chemical agent selected from the group consisting of one or more of glyoxal, 1-Ethyl-3-(3-(dimethylamino) propyl)-carbodiimide (EDC), glutaraldehyde (GA), and oxaldehyde (OA).

16. The process of claim 8, further comprising mixing the nanoporous protein crystals with dimethylamine borane complex (DMAB).

17. A process of making a porous protein crystal drug delivery system, comprising:
preparing protein crystals derived from a polyisoprenoid-binding protein from Campylobacter jejeuni (SEQ ID NO: 14) or a variant of a polyisoprenoid-binding protein from Campylobacter jejeuni (SEQ ID NO: 15), the protein crystals having a particle size ranging from about 100 nm to about 500 nm;
adsorbing a guest macromolecule to the protein crystals; and
capping the protein crystals after adsorbing of the guest macromolecule;
wherein the process of preparing the protein crystals comprises crystallizing a protein sample by a method selected from the group consisting of vapor diffusion and batch crystallization.

18. The process of claim 17, wherein the protein crystals are capped with a protein comprising D2 domain from human N-ethylmaleimidesensitive factor (NSF) (SEQ ID NO: 13).

19. The process of claim 17, wherein the vapor diffusion is selected from one of sitting drop vapor diffusion and hanging drop vapor diffusion.

20. The process of claim 17, wherein the process of preparing the protein crystals further comprises mixing a precipitant with the protein sample, the precipitant comprising ammonium sulfate.

21. The process of claim 17, wherein the protein crystals are crosslinked by a process comprising mixing the protein crystals with a chemical agent selected from the group consisting of one or more of glyoxal, 1-Ethyl-3-(3-(dimethylamino) propyl)-carbodiimide (EDC), glutaraldehyde (GA), and oxaldehyde (OA).

22. The process of claim 21, further comprising mixing the protein crystals with dimethylamine borane complex (DMAB).
